(19) United States
(12) Patent Application Publication
(10) Pub. No.: US 2005/0064525 A1
(43) Pub. Date: Mar. 24, 2005

(54) Method of screening pigs for resistance to salmonellosis

(76) Inventors: Marijke Kreukniet, Bs Vught (NL); Pauline Van Diemen, Berkshire (GB); Nat Bumstead, Berkshire (GB); Janene Bumstead, Compton near Newbury Berkshire (GB); Tim Wallis, Berkshire (GB); Lucina Galina-Pantoja, Hendersonville, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/485,024

(22) PCT Filed: Jul. 30, 2002
(86) PCT No.: PCT/GB02/03465

(30) Foreign Application Priority Data
Jul. 30, 2001 (GB) 0118549.5

(51) Int. Cl. G01N 33/554; G01N 33/569

(52) U.S. Cl. 435/7.32

(57) ABSTRACT

The present invention relates to methods for assessing an animal's susceptibility to bacterial infection. Methods for selecting suitable breeding animals are also provided.

The present invention relates to methods for assessing an animal's susceptibility to bacterial infection. Methods for selecting suitable breeding animals are also provided.

High-density pig rearing poses a higher risk of exposure to potentially lethal infectious agents. Hygienic measures, vaccination and treatment can reduce the effects of diseases, however, the development of livestock with genetically increased resistance to infectious diseases is very desirable, particularly with the emergence of multiple antibiotic resistant zoonotic pathogens (Wall et al., 1995; Van der Wolf et al., 1999).

Host resistance to infection is multifactorial and reflects the highly complex host/pathogen interactions that take place during the infection process. In addition to the virulence of the microbial agents, the efficacy of the host's innate and specific immune defence mechanisms that mediate resistance to infection determine the severity of infection. Genetic variation in immune responsiveness in pigs has been well documented (Kräusslich et al., 1983; Buschmann et al., 1985; Joling et al., 1993; Edfors-Lilja et al., 1994; Magnusson et al., 1997). Little is known, however, about the protective immune responses to specific diseases, making it difficult to predict which host defence mechanism will offer the optimal protection against a particular disease. High levels of immune activity might intuitively seem ideal, but these may give rise to autoimmune disorders or inappropriate responses to particular diseases (Pevzner et al., 1981).

Recently progress has begun to be made in understanding the molecular basis of host resistance to infection. Three major genetic loci that influence host resistance to Salmonella infection have been characterised, two in mice (Nramp1, Vidal et al., 1995; LPS, Ková\check{r}ová et al.,. 1997) and one in poultry (SAL1, Bumstead, unpublished).

The influence of the different genetic loci on host resistance, however, is difficult to predict and requires experimental infection studies to define variation in host resistance. The application of quantitative trait locus (QTL) analysis to defined experimental infection models represents a powerful approach for identifying host resistance genes. Salmonellosis in pigs represents a potentially useful infection model for the study of disease resistance for two reasons. First, the disease is quantifiable. During infection Salmonella invade and translocate across the intestinal mucosa and become systemically disseminated to various organs, including the liver and spleen. By enumerating Salmonella within these organs, the severity of disease can be quantified (Watson et al., 2000). A similar approach was successfully exploited in the early characterisation of the susceptibility of different mouse strains to salmonellosis (Hormaeche, 1979). Second, salmonellosis is a common disease of swine. Salmonella outbreaks and subclinical infections occur in livestock around the world. They are often not only a cause of economic and animal welfare costs, but also a source of contamination of pork products entering the food chain (Baggesen et al., 1996; Van der Wolf et al., 1999; Hamilton et al., 2000; Käisbohrer et al., 2000; Davies et al., 2000). Because the severity of the disease and the concomitant productivity losses in commercial units vary greatly between different herds, a genetic variation in the predisposition of the pig-to salmonellosis seems likely.

The aim of the present study was to assess the degree of resistance to Salmonella choleraesuis infection in a purposely-bred reference family, to facilitate the subsequent mapping of resistance genes. Within this reference family, aspects of the innate and specific immune system of individuals with defined susceptibility to salmonellosis were studied, to identify immune parameter(s) that might predict resistance to salmonellosis in pigs.

We have now identified certain parameters which indicate susceptibilty to bacterial infection.

Thus, in a first aspect the present invention provides a method for assessing an animal's susceptibility to bacterial infection, which comprises measuring at least one immune function of the animal. The immune function can be measured prior to infection. Preferably, prior to infection, the method comprises determining the intrinsic activity of non-stimulated polymorphonuclear neutrophils (PMNs) or measuring lymphocyte proliferation following appropriate stimulation with a mitogen.

Intrinsic activity of the non-stimulated PMNs is suitably measured by means of measuring oxidative burst response.

Lymphocyte proliferation is suitably measured following stimulation by Concavalin A (Con A).

Alternatively the immune function can be measured post infection. Preferably, post infection, the method comprises monitoring the pyrexic response of the animal.

The methods of the invention can also be carried out by determining the number of neutrophils in a sample from the animal.

The method of the invention can also be carried out by measuring the reponse to anti-LPS antibody.

The method of the present invention allows animals to be segregated into “resistant” and “less resistant” groups once an infection has been recognised. This helps to prevent the spread of the infection. This provides the producer with the opportunity to maintain batches of animals that are free of treatments or to produce batches of animals that have a more consist body mass and size.

In particular, the methods find use in assessing susceptibility to Salmonella infection and are particularly suited to assessing pigs.

As described herein, it is important for animal breeders to have reliable methods for selecting suitable animals for breeding programmes. The methods of the present invention provide just such a means. Thus, in a second aspect the present invention provides a method of selecting animals for suitability as breeding animals which comprises carrying out a method of the invention as defined herein.

The invention will now be described with reference to the following examples, which should not be construed as limiting the invention.

### EXAMPLE 1

The reference population was bred from animals derived from 2 commercial pig lines (A, B). Parents were full-sister pairs of F1 crosses between the two lines (line A×B and line B×A) and 4 boars (line A). The boars were selected based on the results of a preliminary study to identify families that differed in susceptibility to infection with Salmonella clioleraesuis. Offspring of the first boar (G398, n=4) was more susceptible to Salmonella than the offspring of the second boar (G402, n=4); they had higher mortality (2 vs 0), higher bacterial recovery counts (liver: 4.97 vs 2.07; spleen: 4.48 vs 0.69 colony forming units expressed as a ¹⁰log-scale), maintained an increased rectal temperature (>40° C.) after the initial fever peak and lost more weight (300 g/d) during the infection week.

On this basis, boar G398 was regarded as susceptible and boar G402 as resistant. Boar Y2008 was a son of G402 and was characterised as ‘possible resistant’, while the 4^{th} boar (Y6101) was related to G398 and was characterised as ‘possibly susceptible’. Table 1 gives the structure of the reference family. The 43 F1-gilts were derived from 19 sows and 7 sires. Twenty-three of the gilts mated to Y2008 and Y6101 had the same sire (B417, half sibling group). Each gilt produced one litter, and between 3 and 9 piglets per litter were randomly selected for this study. A total of 216 piglets, in 12 groups of 18 piglets, were screened for their degree of resistance to infection with S. choleraesuis. Each group compared offspring of 2 boars. Group 1 to Group 5 compared of offspring of G398 and G402; Group 6 to Group 12 compared offspring of Y2008 and Y6101.

TABLE 1
Structure of the reference family: number of piglets
and litters, sex ratio, and mean (SD) age and body weight
(BW₃₀) on the day of infection (Day 30) per boar.

| | Boar | | | | |
|---|---|---|---|---|---|
| | G398 | G402 | Y2008 | Y6101 | Overall |
| n | 47 | 40 | 53 | 64 | 204 |
| litters | 11 | 8 | 11 | 13 | 43 |
| sex ratio (% | 44.7 | 55.0 | 52.8 | 59.4 | 53.0 |
| ♂ of total) | | | | | |
| age (days) | 45.2 (1.0) | 46.0 (1.2) | 43.9 (1.2) | 43.9 (1.3) | 44.6 (1.4) |
| BW₃₀ (kg) | 11.5 (2.5) | 11.6 (2.1) | 11.4 (2.6) | 10.2 (3.0) | 11.1 (2.7) |

Screening of Pigs for Susceptibility to Salmonellosis

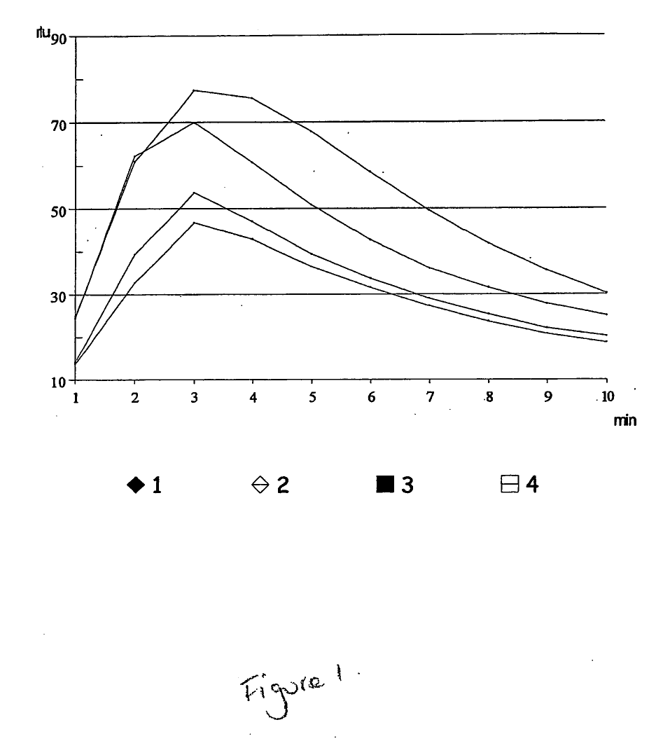

The experimental period was composed of a pre-infection period of 30 days, and an infection period of 7 days. The first day of the pre-infection period was defined as Day 1. On Day 1, the pigs (101 ♀♀ and 115 ♂♂) were on average 13.6±1.4 days old and weighed 3.96±0.8 kg. FIG. 1 gives a schematic survey of the experimental routine per group.

At three-week intervals, the groups of gilts farrowed in an outdoor facility at IAH, Compton. At 2 weeks of age, selected piglets were moved to the Medium Security bio-containment unit (MSU) at IAH, where they were treated with Baytril® 2.5% on three consecutive days (0.5 ml im of 25 mg/ml enrofloxacin, Bayer plc, Suffolk, UK). The piglets were housed in Nürtinger Units (9 pigs/unit) and fed on evaporated milk twice daily and a pelleted Salmonella-free (irradiated) diet (Vitastart, Denkavit, Walsingham, UK) ad libitum by self-feeders with free access to water. After 2 weeks, piglets were moved to floorpens with free access to Salmonella-free food (Vitalink, Denkavit, Walsingham, UK) and water. Normal medication of the feed was stopped and the Cu level in the feed was lowered to the nutritional level of 35 mg/kg. To eliminate bacterial contamination, all diets were exposed to a minimum irradiation dose of 10 kGy.

Upon entry to the MSU and 4 weeks later, pigs were checked for the absence of Salmonella by enrichment cultures of rectal swabs. Two days before the challenge (Day 28) the pigs were transferred to High Security bio-containment accommodation and housed in tanks on tenderfoot mats (3 pigs/tank). On day 31, pigs were challenged ora/gastrically with ˜8×10⁸ cfu S. choleraesuis in 10 ml antacid (0.5 g MgO, 0.5 g Mg trisilicate, and 0.5 g NaHCO₃ in H₂O). The bacterial strain used was S. choleraesuis var. kunzendorf A50, a well characterised strain of defined virulence for pigs (Watson et al., 2000). Rectal temperatures were recorded twice daily, starting on the day before challenge till the end of the experiment. Daily weight gain (g/d) was calculated over day 1-7 (adaptation period), day 7-30 (pre-infection period), and day 30-38, the infection period, which was split into ‘early’ infection (day 30-35) and ‘late’ infection (day 35-38). Seven days after challenge pigs were necropsied (stunned and bled) and post mortem triplicate samples of liver and spleen were taken to determine bacterial recoveries. Earnotches were collected and stored at −20° C. to facilitate prospective mapping of resistance genes.

Animals which approached humane endpoints were killed with 10-15 ml Euthatal iv (200 mg/ml Pentobarbitone Sodium BP, Rhône Mérieux, Essex, UK). Post mortem samples were taken. Endpoints were a combination of lethargy, anorexia (weight loss >25% of body weight at infection) and/or if malaise and dehydration result in the pig being unable to stand unaided.

The number of viable Salmonella per gram of liver and spleen were enumerated by grinding 1 g of tissue in 9 ml 0.9% NaCl (in triplicate), plating 100 μl aliquots (triplicates) onto Brilliant Green agar (BGA, Difco 1880), incubating for ˜18 hrs and counting the number of viable S. choleraesuis colonies. Bacterial recoveries were analysed on a ¹⁰log-scale; Detection limit 2 logs per plate or 0.22 logs per-tissue (3×3 plates/tissue).

Blood and Immune Characteristics

Blood samples were drawn for serum analyses before infection (Day 29), and on the post mortem day (Day 38). Heparinised blood samples were drawn on Day 15, Day 29 and on the post mortem day (Day 38). Heparinised blood samples were analysed for the numbers of white blood cells (WBC) per ml with a Coulter Counter® Z1 (Coulter Electronics LTD, Luton, UK). Proportions of lymphocytes, neutrophils, monocytes and eosinophils were determined by counting and classifying one hundred leukocytes after making a blood smear, stained with Hemacolor® rapid staining (Merck, Darmstadt, Germany), and total numbers per ml were calculated by multiplying the proportions by the WBC count.

Polymorphonuclear neutrophils (PMNs) were isolated from heparinised blood samples using Ficoll density gradient centrifugation (Polymorphprep™, Nycomed Pharma AS Diagnostics, Oslo, Norway) and resuspended in modified Hank's medium (Vassiloyanakopoulos et al., 1998). On day 15, the oxidative burst of PMN was determined in vitro by Lucigenin enhanced chemiluminescence (Aniansson et al., 1984). In 96-well optiplates (Hi-White-TC optiplate 1, LabTech International, Ringmer, UK), in triplicate, cell suspensions (5×10⁴/well) were mixed with either modified Hank's medium (background, intrinsic activity), opsonised Zyrnosan (Sigma, positive control), opsonised S. choleraesuis, or Zymosan with S. choleraesuis, all in the presence of Lucigenin (bis-N-Methylacridinium Nitrate, Sigma). In a microplate luminometer (MicrolumatPlus, EG&G Berthold, Milton Keynes, UK), the kinetics of the respiratory burst during the phagocytosis process were recorded approximately every 4 min. (2 sec/well) during a 45 min. incubation period at 37° C. The oxidative burst by PMNs after uptake of zymosan particles estimates the maximal activated state of these cells, which can be compared with the burst induced by Salmonella.

The in vitro bactericidal capability of pig PMNs was tested on day 29, using an adaptation of the method described by Vassiloyanakopoulos et al. (1998). For the in vitro phagocytosis and intracellular killing of S. choleraesuis, the PMNs under test (1×10⁶) were incubated (shaking) for 15 min. at 37° C. with ˜7.5×10⁶ cfu S. choleraesuis in 1 ml modified Hank's medium supplemented with 5% normal pig serum. The cultures were washed and resuspended in modified Hank's medium with gentamicin to kill extracellular bacteria. The sample was split; One part, to quantify phagocytosis of Salmonella, was washed again to remove the gentamicin, PMNs were lysed with ice-cold water, and 100 μl of ten-fold dilutions (10⁻⁰ to 10⁻³) of the lysed PMNs suspension were plated on Brilliant Green agar (BGA) plates. The other part, to quantify killing of Salmonella, was incubated for another 60 min., after which the cells were washed and lysed, and aliquots (neat) plated on BGA. After 18 hours incubation at 37° C., Salmonella colonies were counted. From the number of viable intracellular bacteria, the number of phagocytosed bacteria per PMN, and the proportion of subsequently killed bacteria were calculated.

On days 29 and day 38, S. choleraesuis-specific cellular immunity, and mitogenic proliferation was determined in vitro by means of a whole blood lymphocyte proliferation assay in flat-bottom microplates. Ten percent (v/v) heparinised whole blood samples were tested for white blood cell proliferation in the presence of either 5 μg/ml Concanavalin A (ConA), 5 μg/ml Whole Cell Protein of S. choleraesuis (WCP, Hassan et al., 1990), 5 μg/ml heat extract antigen of S. choleraesuis (H E, Grey et al., 1995) or 5 μg/ml ConA with 1:20 diluted S. choleraesuis LPS in RPMI culture medium containing antibiotics. All cultures, set up in triplicate, were incubated for 4 days at 37° C., 5% CO₂ in a humidified atmosphere. Eighteen hours before cells were harvested, 0.4 μCi of methyl-³H-thymidine (³H, Amersham, Bucks, UK) was added. The ³H uptake was determined with a Beckman β-scintillation counter. Results are expressed as mean counts per minute (cpm). Stimulation index (SI) was calculated as: SI=cpm in antigen stimulated cultures/cpm in unstimulated cultures. A SI>2 was regarded as positive.

Sera collected on day 29 (pre-infection) and on day 38 (post mortem) were analysed with the Danish mix-ELISA (Nielsen et al., 1995). This ELISA contains a mix of LPS from S. typhimurium O: 1,4,5,12, S. infantis O: 6,7 and S. choleraesuis var. Kunzendorf O: 6,7 as antigen. Diluted sera were added to the antigen-coated wells. After incubation for 1 h at room temperature and subsequent washing, samples were incubated with horseradish peroxidase-conjugated rabbit antiserum to swine Ig (DAKO). After another washing step H₂O₂/OPD substrate was added. The optical density (OD) was read at 490 nm with background correction of 650 nm. ODs are expressed as a percentage of a positive reference serum defined as 100%, corrected for non-specific binding using a negative reference serum. On this basis Salmonella negative pigs have an OD value of 0% (Nielsen et al., 1994).

Statistical Analysis

Data on weight gain, recoveries of Salmonella from liver and spleen (post mortem), and blood and immune characteristics were statistically analysed for effect of Group, Boar and their interaction using a two-way analysis of variance with ‘Dam within Group and Boar’ as random effect (Proc Mixed, SAS, 1995). Groups 1-5 and Groups 6-12 were analysed separately. When appropriate, comparisons were made between the four groups of pigs as characterised by their father (boar). The effects of Group, Boar and their interaction on parameters measured twice or more (rectal temperature, WBC, lymphocytes, neutrophils, oxidative burst) were tested by means of an F-test, with the data taken as repeated measurements. For the analyses of stimulation indices, the number of lymphocytes was included in the model as a covariable. Subsequently, pairwise comparisons between boars were performed at the overall 0.05 level of significance. Relationships between the traits were orthogonally fitted by polynomial regression using Proc Mixed and Proc GLM (SAS, 1995).

Results

All pre-infection enrichment cultures from rectal swabs were negative for Salmonella. Table 1 gives a summary of the selected piglets per boar on day 30. Seven piglets suffered from diarrhoea in the pre-infection period and were excluded from the study. Thus, a total of 204 piglets were challenged with S. choleraesuis. The infection dose ranged between 4.5×10⁸ and 1.4×10⁹ cfu, for the different groups.

The number of pigs reaching the predetermined humane clinical endpoint (prior to 7 days post infection) was significantly higher in the latter 7 groups (33% of the Y6101-offspring and 23% of the Y2008-offspring) than in the first 5 groups (1 animal from each boar). Two types of end-points were found; acute after onset of the disease (day 2/3) and peracute, day 5/6 post infection (Table 2).

The 33 pigs that reached their endpoint prior to the scheduled post mortem day of Groups 6-12 affected to some extent the results of all parameters measured after infection. By taking out the most susceptible pigs, the post mortem day results are expected to be skewed towards the more ‘resistant’ animals.

Bacterial Recoveries

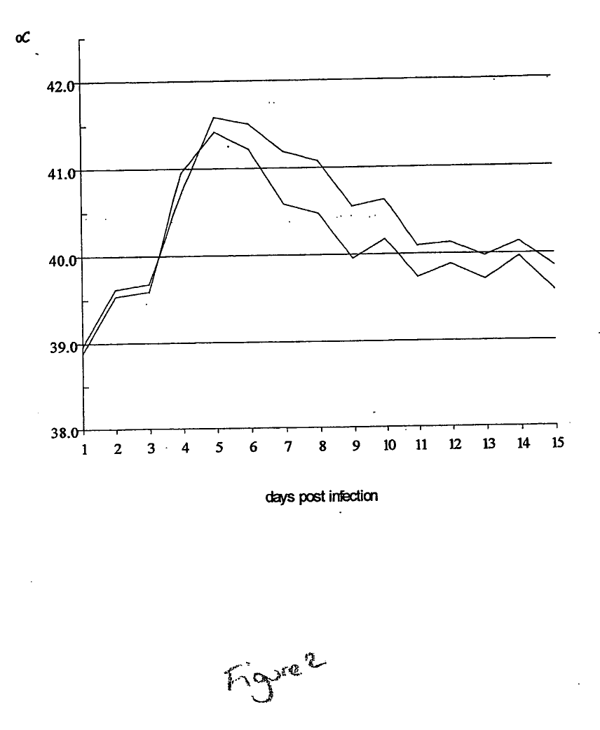

The number of bacteria recovered from both liver and spleen in groups 1-5 show that the G398-offspring was significantly more susceptible to the infection with S. choleraesuis than the G402-offspring (FIG. 2). Significant Group and Sex effects were found on the recoveries. Interactions between these fixed effects and the effect of Boar, however, were not significant. Males had lower recoveries than the females (liver: 1.11 vs 1.54, P<0.04; spleen: 0.40 vs 0.69, P<0.08).

The offspring of the boars Y6101 and Y2008 did not differ from each other but higher numbers of bacteria were recovered both from liver and spleen compared with the offspring of G398 and G402 (FIG. 2). The pigs that reached the clinical endpoint before day 7 p.i., in general, had higher number of bacteria in both liver and spleen than the pigs that were killed on day 7 p.i. (liver: 3.8 vs 2.4, and spleen: 2.5 vs 1.1). Bacterial recoveries from liver and spleen were highly related to each other (r²=0.78); higher recoveries from spleen were associated with higher recoveries from liver. This relationship was similar in all offspring groups.

Pyrexic Response

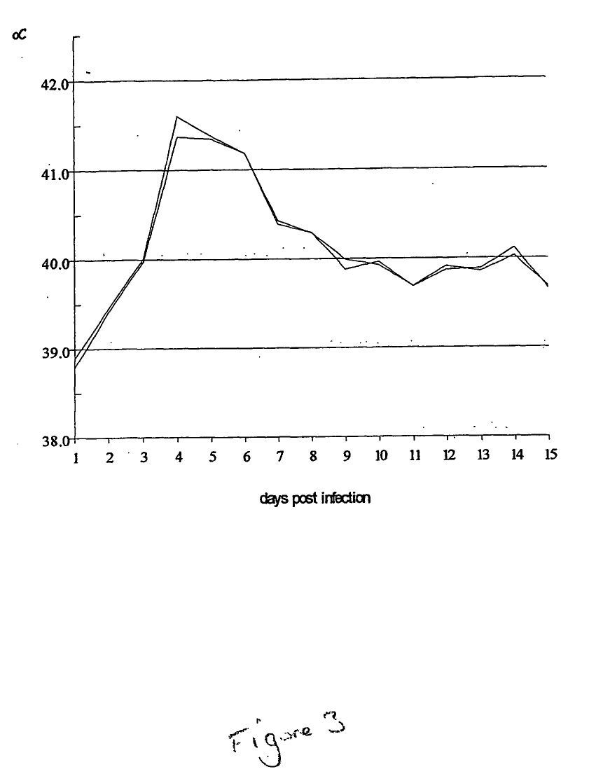

On average over the infection period the G398 pigs had a significant higher rectal temperature than the G402 pigs (0.27° C., P<0.001). These 2 offspring groups showed a significantly different pattern in rectal temperature over time (FIG. 3). The G398 pigs showed a slower increase (P<0.05) towards a slightly higher fever peak (0.16° C., P=0.1) on day 2 post infection. After the peak, the temperature of the G402 pigs dropped to almost normal (40° C.) on day 4, while the temperature of the G398 piglets dropped at a significantly slower rate compared with the G402 pigs.

The offspring of Y2008 and Y6101 showed a fast pyrexic response (FIG. 3) with the peak significantly higher for the Y2008 pigs (0.23° C., P<0.05), at the end of day 1 post infection. After this point, however, the most susceptible animals reached the humane clinical endpoint and had to be culled (Table 2).

TABLE 2
Number of endpoint pigs per boar per day post infection.

| | | Endpoint on day post infection | | | | |
|---|---|---|---|---|---|---|
| Boar | n/total | 2 | 3 | 4 | 5 | 6 |
| G398 | 1/47 | | 1 | | | |
| G402 | 1/40 | | | | | 1 |
| Y2008 | 12/53 | 3 | 4 | 0 | 1 | 4 |
| Y6101 | 21/64 | 5 | 6 | 1 | 4 | 5 |
| | 35/204 | 8 | 11 | 1 | 5 | 10 |

Performance

The mean initial body weight and the body weight just before infection (BW₃₀, Table 1) of the piglets were similar for all 4 boars, but varied between groups and litters. In general, the piglets grew more slowly, or lost weight during the first 4-5 days post infection. Most animals in Groups 1-5 (74/85) returned to gaining weight during the later phase of infection. The weight gain of the G402 pigs during infection was on average 100 g/d better than of the G398 progeny (P<0.05). In Groups 6-12, at least half of the pigs continued to lose weight over the later phase of infection. The challenge had a larger impact on the growth of Y2008 (−106 g/d, sem 23.6) and Y6101 (−151 g/d, sem 21.8) offspring than of the G398 (+76 g/d, sem 27.9) and G402 (+176 g/d, sem 31.4) offspring.

Blood and Immune Characteristics

The number of white blood cells (WBC) per ml increased significantly (P<0.05) during the infection week in all offspring groups. On day 38 (day 7 post infection), the WBC was significantly higher for the G402 offspring than for the G398 offspring (2.2×10⁶/ml). The offspring of the 2 other boars did not differ from each other before challenge or at the end of the experiment. Both the lymphocyte and the neutrophil counts increased during infection, significantly more in the G402 offspring than in the G398 offspring, resulting in a significant difference at day 38 for both cell types of ˜1.1×10⁶/ml (Table 3). The lymphocyte and neutrophil counts of the Y2008 and Y6101 offspring did not differ from each other. Both counts increased significantly after infection (P<0.05). The results per offspring group are summarised in Table 3. Group had a significant effect on all the blood and immune parameters measured, but no interaction with Boar was found.

The oxidative burst of PMN induced with the different antigens was significantly affected by Group and Group-time interaction. The groups differed in magnitude of the oxidative burst, though the kinetics were quite similar. The intrinsic activity of non-stimulated PMN differed between the offspring of G398 and G402, the latter having a higher activity (FIG. 4). Only in the first 5 groups, Salmonella caused an oxidative burst slightly higher than the intrinsic response. The maximal activated state of the PMN induced by uptake of zymosan particles did not differ between the offspring of G402 and G398. Co-stimulation with Salmonella reduced the magnitude of the zymosan induced oxidative burst, but did not differ between the offspring groups. Y6101 piglets had a somewhat higher (P<0.1) zymosan induced burst than the Y2008 pigs. However, adding Salmonella to the cultures did not influence this response.

The PMN originating from the G402 piglets phagocytosed more Salmonella (P<0.04) and killed them more efficiently (P<0.08) than those from G398 pigs (Table 3). No difference was found between Y2008 and Y6101 offspring.

The proliferative capacity of lymphocytes (PBL) from pigs in the reference family both pre- and post infection was assessed following non-specific and specific immunostimulation. The proliferations of PBL stimulated with ConA and with ConA combined with LPS are given in Table 3. Cpm of unstimulated cultures averaged 163 before infection, and averaged 203 after infection. The PBL of the G398 pigs showed a stronger ConA proliferation response than the G402 offspring, both before and a week after infection. The. Y6101 pigs had a significantly higher proliferative response to ConA than the Y2008 pigs. The proliferation of PBL to ConA combined with LPS was significantly inhibited compared with the ConA alone (>60%, Table 3). This inhibition was significantly higher post infection in the G402 offspring, while the infection did not change this response in the G398 pigs. In the latter groups, it was the other way round, the LPS induced inhibition was significantly higher before infection than after in the Y6101 pigs, while the Y2008 offspring kept the same lever of inhibition pre- and post infection.

TABLE 3
Least square means (SEM) and significance level of blood and immune characteristics
as affected by Boar on the different testdays.

| | |
|---|---|
| Group 1-5 | Group 6-12 |

| | Day | G398 | G402 | sem | P-value¹ | Y2008 | Y6101 | sem | P-value¹ |
|---|---|---|---|---|---|---|---|---|---|
| WBC × 10⁶/ml | 29 | 8.78 | 9.17 | 0.44 | 0.58 | 5.91 | 6.09 | 0.25 | 0.68 |
| | 38 | 9.75 | 11.96 | 0.60 | 0.02* | 10.72 | 9.95 | 0.66 | 0.43 |
| lymphocytes × 10⁶/ml | 29 | 5.11 | 5.38 | 0.31 | 0.35 | 3.28 | 3.51 | 0.20 | 0.49 |
| | 38 | 5.10 | 6.16 | 0.31 | 0.02* | 4.28 | 4.35 | 0.23 | 0.85 |
| neutrophils × 10⁶/ml | 29 | 3.24 | 3.36 | 0.25 | 0.30 | 2.30 | 2.35 | 0.22 | 0.87 |
| | 38 | 4.25 | 5.30 | 0.25 | 0.003** | 5.95 | 5.40 | 0.26 | 0.36 |
| Uptake (n/PMN) | 29 | 0.20 | 0.44 | 0.08 | 0.04* | 0.37 | 0.33 | 0.07 | 0.67 |
| Killing (%) | 29 | 76.0 | 89.6 | 5.06 | 0.08 | 81.83 | 81.89 | 2.70 | 0.98 |
| ConA (SI) | 29 | 184 | 98 | 20.6 | 0.01** | 192 | 322 | 32.4 | 0.01** |
| | 38 | 110 | 54 | 19.0 | 0.09 | 43 | 69 | 9.4 | 0.07 |
| ConA/LPS (SI) | 29 | 53.4 | 29.8 | 9.1 | 0.10 | 34.1 | 50.7 | 5.4 | 0.05* |
| | 38 | 27.1 | 8.2 | 7.0 | 0.16 | 8.9 | 21.6 | 3.7 | 0.03* |
| anti-LPS ELISA | 29 | −2.79 | −1.91 | 2.15 | 0.67 | 2.02 | 1.21 | 1.76 | 0.74 |
| (OD %) | 38 | 31.96 | 24.33 | 2.15 | 0.01** | 37.95 | 35.97 | 2.01 | 0.49 |

¹The effect of Boar on the depicted traits is shown.

Proliferation of PBL in response to the S. choleraesuis antigens (HE and WCP) was not different between the offspring groups. Both HE and WCP induced a mitogenic response, which was not different before or after infection (data not shown).

A week after infection, the G398 piglets mounted a significant higher humoral immune response to the S. choleraesuis LPS than the G402 pigs. This seemed especially true in the Group 5-pigs. In the latter 7 groups, no differences were found between the Y2008 and Y6101 offspring. Group significantly affected the level of anti-Salmonella antibody, but there was no evidence of interaction between Group and Boar.

Correlations

Relationships between the bacterial recoveries and several blood and immune parameters were estimated. Except for the relationship between recoveries from liver and spleen as described above, no strong correlations were found. A smaller but statistically significant association between LPS-inhibition of the ConA response on the one hand and the bacterial recovery on the other was detected (r²=0.30). The higher the % LPS induced inhibition of the ConA response, the lower the recoveries from liver and spleen.

Discussion

This study revealed phenotypic differences with respect to susceptibility to salmonellosis and functional differences in neutrophils and lymphocytes, between the G398- and G402-offspring. These differences were not detected between the Y2008 and Y6101 offspring, however, the survival rate of these pigs was different. Twenty three percent of the Y2008 (G402-son) and 33% of the Y6101 offspring reached the predetermined humane clinical endpoint before the end of the experiment and had to be culled. Such animals must be considered the most susceptible offspring of Y2008 and Y6101, which were lost from the experimental system. Therefore, the Salmonella counts from these offspring groups are skewed as they are derived from the more ‘resistant’ animals.

Comparing G398 and G402 with the younger boars Y6101 and Y2008 shows that average bacterial recoveries were higher in the progeny of the younger boars irrespective of the infection dose. Seasonal factors may have had an influence since challenges took place over a 9-month period. This influence contributed to the significant Group effects. The gilts mated to the younger boars (Y2008 and Y6101) might have been more susceptible; all of them—except one—had the same father, B417. It appears that B417 contributed susceptible alleles, making the majority of his offspring (and grandoffspring).more susceptible. The same applies for the younger boars. Both Y6101 and Y2008 may have received susceptible genes from their parents. The only gilt not sired by B417 had G402 as father and was mated with Y6101. Five of her 7 tested offspring were the most resistant of the group (spleen: 0.58 vs. 2.32, and liver 1.52 vs. 3.21), the other 2 reached their endpoint on day 2 p.i. Although this litter consisted of only 7 pigs, segregation of alleles along the 2 heterozygous parents route with a major dominant locus (R) could be explained by: Rr×Rr→25% RR, 50% Rr and 25% rr.

In the response to the infection, all pigs increased their total number of white blood cells, which was largely due to increased numbers of neutrophils. Neutrophils are one of the innate host defence cell types recruited to the gut when Salmonella invade intestinal mucosa (Wallis et al., 1989). The numbers of neutrophils in the blood of ‘resistant’ and ‘susceptible’ pigs before infection were not different. However, function was significantly different in the cells derived from the offspring of resistant G402 and susceptible G398. The phagocytic capacity and the bactericidal activity of the PMN of the G402 offspring were significantly higher compared with their contemporaries. Also the intrinsic activity of the non-stimulated PMN of G402 offspring was higher, however, the maximal activated state of the PMN (zymosan activation) and the burst induced by Salmonella, were similar between the 2 offspring groups. The younger boars' progeny showed no clear difference in the PMN functions, except for the higher maximal activated state of the PMN of the Y6101 piglets. The differences seen between the progeny of G402 and G398 suggest genetic differences in the phagocytic capacity of PMN and the total number white bloodcells, which are known to have quite high heritability (h²=0.3-0.4, Edfors-Lilja et al., 1994).

In this resource population of pigs, susceptibility to salmonellosis seems to be associated with deficient PMN function under normal conditions and lower PMN numbers post infection. A similar association is seen in susceptibility to E. coli mastitis in sows (Löfstedt et al., 1983). The sows that developed mastitis after inoculation had significant fewer PMN in the peripheral blood and those PMN had depressed function as compared with PMN from sows, which were inoculated, but did not develop mastitis. Foster et al. (2001) showed that systemic infection by virulent Salmonella strains could be prevented in 7-day old gnotobiotic pigs by pre-colonisation of the gut with a non-virulent S. infantis strain. The rapid innate protection against S. typhimurium and S. choleraesuis was stimulated by S. infantis-induced IL-8, causing PMN transepithelial migrations (Foster et al., 2001).

The Nramp1 gene, also known as the Ity locus, controls innate defence to infection, and susceptibility in mice is associated with a single G169D substitution within the fourth predicted transmembrane domain of Nramp1 (Vidal et al., 1995). This particular polymorphism at position 169, however, was not detected in any of the 4 boars used in this study (data unpublished). Van Dissel et al. (1986) described that blood granulocytes of susceptible mouse strains (Ity^{S}) had a two times lower rate of intracellular killing of ingested Salmonella typhimurium than that of resistant mouse strains (Ity^{R}). Mice with wild type Nramp1 need PMN to defend against systemic salmonellosis (Vassiloyanakopoulos et al., 1998). Thus, the recruitment and function of PMN in controlling invasive Salmonella may also be influencial in the innate resistance of pigs to salmonellosis. The relationship(s) between susceptibility to Salmonella infection and porcine PMN function deserve further attention.

The ability of (T) lymphocytes to proliferate when incubated with either a mitogen (non-specific) or an antigen (specific) is an in vitro measure of potential cellular immune response. The Salmonella antigens (whole cell preparations) appeared mitogenic for non-immune quiescent PBL of a large proportion of the piglets, but the response did not differ before or after the infection. The 7 days of infection appear to have been too short to mount a measurable specific cellular immune response. Yet, within this 7-day period there seems to be 2 critical periods for survival (Table 2), corresponding to the course of a typical acute infection. Early on, the cells of the innate immune system have a critical role in controlling invading micro-organisms and play a crucial part in the initiation and subsequent direction of adaptive immune responses. There is a delay of 4-7 days before the initial adaptive immune response takes effect. A proportion of very susceptible pigs die before this specific immune response is mounted, indicating the importance of innate immune mechanisms in controlling the invading Salmonella during this period.

The mitogenic proliferation of lymphocytes in the presence of ConA was significantly higher in the G398 and Y6101 offspring, than in their contemporaries. Mitogenic responses are influenced by intrinsic factors such as variation in number of reactive cells between pigs and genetic variation, and by biological effects such as seasonal influence (Joling et al., 1993). To minimise the effect of test day as a source of variation obscuring influence of pedigree, offspring of the 2 boars within a group were tested on the same day and the data of Group 1 to 5 and Group 6 to 12 were analysed separately. Thus, possible seasonal influence in our experiments is included in the Group effect and could be the cause of part of the difference found between the offspring of the younger and older boars. ConA induced proliferation of PBL has medium high heritability (Edfors-Lilja et al., 1994), and has been used as an indicator in selection for general immune competence of swine (Buschmann et al., 1985; Groves et al., 1993).

The lectin-induced mitogenic response (ConA) a week after infection was significantly reduced compared with before infection. Salmonella-induced immunosuppression of the ConA response has been reported in mice (Matsui and Arai, 1994), chickens (Hassan and Curtiss III. 1994) and pigs (Gray et al., 1996; Kreukniet, personal communication). Matsui and Arai's results suggested that the suppression of T cell proliferation induced by a soluble Salmonella fraction is associated with inhibition of IL-2 secretion and the subsequent response of T cells to IL-2. In our study Salmonella-LPS added to the ConA cultures reduced the proliferative response both before and after infection, and the degree of inhibition was negatively related (r²=0.30) to the bacterial recoveries suggesting a role for LPS in the reduced immune response. Suppression of the immune system could exacerbate Salmonella infection, however, the cells of the most resistant pigs in this study (G402 offspring) had on average the lowest potential to proliferate. Further studies into the connection between proliferative capacity of lymphocytes of an individual with its resistance to salmonellosis are necessary to assess the use of these parameters as a screen for Salmonella resistance.

All pigs mounted an antibody response to Salmonella LPS a week post infection. Given the kinetics of the antibody formation, this is most likely an IgM response. Grey et al (1996) reported a similar IgM response of swine to Salmonella soluble antigen after exposure to S. choleraesuis. In this study, the most resistant pigs (G402 offspring) had the lowest antibody response. The younger boars' progeny had an even higher humoral response than the G398 offspring, but this higher humoral response of the G398, Y2008 and Y6101 pigs was not protective in the early stages of the Salmonella infection.

In summary, piglets with different susceptibilities to Salmonella choleraesuis infections were identified. The most resistant piglets (G402) had a higher number of neutrophils and a better PMN function, but a lower mitogenic response of lymphocytes both pre- and post-infection and a lower antibody response.

Conclusion

The reference family has been successfully built. Our findings suggest a role for several hereditary determined parameters, including PMN function and lectin-induced mitogenic proliferation, that could influence resistance to salmonellosis. Adequate innate immune mechanisms are of importance for pigs to survive the first week after exposure to Salmonella. The results of Groups 1-5 (G398 and G402 offspring) particularly indicate a genetic element in resistance to salmonellosis that is inherited through families. DNA samples were prepared from all pigs involved in this study and given the significant differences in susceptibility, the number of pigs in this study could be sufficient for successful mapping of candidate host resistance genes.

1. a method for assessing an animal's susceptibility to bacterial infection, which comprises measuring at least one immune function of the animal:

2. A method as claimed in claim 1, wherein the immune function is measured prior to infection.

3. A method as claimed in claim 2 wherein the method comprises determining the intrinsic activity of non-stimulated polymorphonuclear neutrophils (PMNs).

4. A method as claimed in claim 2 wherein the method comprises measuring lymphocyte proliferation following appropriate stimulation with a mitogen.

5. A method as claimed in claim 3 wherein the intrinsic activity is measured by means of measuring oxidative burst response.

6. A method as claimed in claim 4 wherein lymphocyte proliferation is measured following stimulation by Concavalin A (Con A).

7. A method as claimed in claim 1, wherein the immune function is measured post infection.

8. A method as claimed in claim 7, which comprises determining the number of neutrophils in a sample from the animal.

9. A method as claimed in claim 7, wherein the method comprises measuring the response to anti-LPS antibody.

10. A method as claimed in claim 7, wherein the method comprises monitoring the pyrexic response.

11. A method as claimed in any one of claims 1 to 10 wherein the assessment is carried out in respect of Salmonella infection.

12. A method as claimed in claim 1 wherein the assessment is carried out on a blood sample obtained from the animal.

13. A method of selecting animals for suitability as breeding animals which comprises carrying out a method as defined in claim 1.

14. A method as claimed in any one of claim 1 wherein the animal is a mammal.

15. A method as claimed in claim 1 wherein the animal is a pig.
